(19) United States
(12) Patent Application Publication
(10) Pub. No.: US 2026/0305956 A1
(43) Pub. Date: Oct. 8, 2026

(54) System for Obtaining Diagnostic Data Indicative of a Cardiac Condition of a Patient

(71) Applicant: BIOTRONIK SE & CO. KG, Berlin (DE)

(72) Inventors: Ravi Kiran Kondama REDDY, Portland, OR (US); R. Hollis WHITTINGTON, Portland, OR (US); Daniel YOUNG, Portland, OR (US); Patrick L. PARISH, Portland, OR (US)

(73) Assignee: BIOTRONIK SE & CO. KG, Berlin (DE)

(21) Appl. No.: 19/482,088

(22) PCT Filed: May 15, 2024
(86) PCT No.: PCT/EP2024/063299

§ 371 Date: Nov. 6, 2025

Related U.S. Application Data
(60) Provisional application No. 63/467,718, filed on May 19, 2023.

(30) Foreign Application Priority Data
Jun. 14, 2023 (EP) 23179139.3

(51) Int. Cl. A61B 5/0205; A61B 5/00; A61B 5/026; A61B 5/0531; A61B 5/107; A61B 5/283; A61B 5/346

(52) U.S. Cl. CPC A61B 5/02055; A61B 5/0024; A61B 5/0031; A61B 5/0053; A61B 5/346; A61B 5/6831; A61B 5/026; A61B 5/0531; A61B 5/1073; A61B 5/283

(57) ABSTRACT

includes an implantable medical device including a sensing arrangement for sensing a cardiac diagnostic signal, a sensor apparatus configured to be placed on skin tissue on a patient's extremity and including at least one sensor device for sensing a remote diagnostic signal on the skin tissue, and a processing arrangement for processing data relating to said cardiac diagnostic signal and said remote diagnostic signal to obtain diagnostic data indicative of a cardiac condition.

### CROSS-REFERENCE TO RELATED APPLICATIONS

This application is the United States National Phase under 35 U.S.C. § 371 of PCT International Patent Application No. PCT/EP 2024/063299, filed on May 15, 2024, which claims the benefit of European Patent Application No. 23179139.3, filed on Jun. 14, 2023, and U.S. Provisional Patent Application No. 63/467,718, filed on May 19, 2023, the disclosures of which are hereby incorporated by reference herein in their entireties.

### TECHNICAL FIELD

The instant invention relates to a system for obtaining diagnostic data indicative of a cardiac condition of a patient, and to a sensor apparatus configured to be placed on skin tissue on a patient's extremity.

### BACKGROUND

The system generally comprises an implantable medical device comprising a sensing arrangement for sensing a measurement signal indicative of a cardiac function.

An implantable medical device of the type concerned herein generally may for example comprise an arrangement of electrode poles configured to at least one of output electrical stimulation signals for effecting a stimulation action or sense electrical excitation signals.

The implantable medical device may for example be a cardiac pacemaker, an implantable cardioverter defibrillator, a sensor device such as a bio-sensor, or a monitoring device. The implantable medical device may serve a cardiac diagnostic and/or therapeutic function.

For example, the implantable medical device may be a cardiac monitoring device which is configured to record electrocardiogram signals and to communicate recorded electrocardiogram signals or information derived from recorded electrocardiogram signals to an external device in the context of a home monitoring system.

An implantable medical device as for example described in EP 3 278 836 B1 may for example comprise a housing and an arrangement of electrode poles arranged on the housing. The electrode poles herein are arranged on the housing of the implantable medical device such that the electrode poles are aligned along a longitudinal axis along which the implantable medical device extends. The electrode poles may for example be formed by housing segments which are made from an electrically conductive material such as a metal material and are exposed to the outside such that they may be brought into electrical contact with surrounding tissue in order to establish an electrical coupling to the tissue in an implanted state of the implantable medical device.

Measurement signals which are sensed by the implantable medical device may for example relate to electrocardiogram signals. In other embodiments, measurement signals may relate for example to a respiratory function.

Generally, a cardiac disease, such as a chronic deterioration of a cardiac condition, in particular a chronic heart failure, has been shown to correlate with peripheral edema in extremities of the patient, in particular the lower limbs. Therefore, detecting edema may provide for diagnostic data which may facilitate the diagnosing of a cardiac disease and the identification of e.g. a heart failure condition.

In known clinical approaches, in order to determine the presence of fluid/edema in skin tissue of a patient a so-called pitting test is performed, during which a physician manually presses on the skin surface with a finger for example for a few seconds and observes whether a dimple in the skin tissue starts to form. Depending on the depression depth and depending on how long it lasts for the skin tissue to reset to its nondeformed state, a score between 0 (no edema) to 4 (most severe edema) is assigned. As fluid retention has been postulated to be responsible for a clinical manifestation of a heart failure condition, an edema score may be taken into account when diagnosing a patient for a heart failure condition.

The present disclosure is directed toward overcoming one or more of the above-mentioned problems, though not necessarily limited to embodiments that do.

### SUMMARY

It is an object to provide a system for obtaining diagnostic data indicative of a cardiac condition of a patient as well as a sensor apparatus which allow to obtain additional diagnostic information, beyond the information which may be obtained with an implantable medical device, to facilitate the diagnosing of a heart failure condition.

In one aspect, a system for obtaining diagnostic data indicative of a cardiac condition of the patient comprises an implantable medical device comprising a sensing arrangement for sensing a cardiac diagnostic signal, a sensor apparatus configured to be placed on skin tissue on a patient's extremity and comprising at least one sensor device for sensing a remote diagnostic signal on the skin tissue, and a processing arrangement for processing data relating to the cardiac diagnostic signal and the remote diagnostic signal to obtain diagnostic data indicative of a cardiac condition.

The implantable medical device may for example be a cardiac pacemaker, an implantable cardioverter defibrillator, a sensor device such as a bio-sensor, or a monitoring device. The implantable medical device may serve a cardiac diagnostic and/or therapeutic function. The implantable medical device may in particular be configured, with its sensing arrangement, to sense electrical signals in order to obtain electrocardiogram signals indicative of a cardiac functioning. For this, the sensing arrangement may for example comprise a multiplicity of electrode poles, placed on a housing of the implantable medical device and/or on one or multiple leads extending from the implantable medical device to reach into the patient's heart or to extend outside of the patient's heart.

The implantable medical device, with its sensing arrangement, senses measurement signals indicative of a cardiac function, for example electrocardiogram signals. In addition, the system comprises a sensor apparatus configured to be placed on skin tissue on a patient's extremity, for example or on a lower limb of the patient, e.g. on a foot of the patient, preferably near the ankle of the foot. The sensor apparatus is operated in conjunction with the implantable medical device, wherein a processing arrangement processes cardiac diagnostic signals as obtained by the implantable medical device and remote diagnostic signals as obtained by the sensor apparatus. Information obtained according to cardiac diagnostic signals from the implantable medical device and information obtained according to remote diagnostic signals from the sensor apparatus hence may be combined in order to facilitate a diagnosing of a cardiac condition, in particular a heart failure condition.

By including a sensor apparatus remote from the implantable medical device into the system, the sensor apparatus being configured for placement on skin tissue on a patient's extremity, additional diagnostic data may be obtained and may be combined with cardiac diagnostic signal data, in particular data relating to electrocardiogram signals, as generally obtained using an implantable medical device serving a cardiac diagnostic and/or therapeutic function. By combining data from the implantable medical device and from the sensor apparatus to be operated remote from the implantable medical device on skin tissue on a patient's extremity, a diagnosis and treatment of a heart failure condition may be facilitated in that additional diagnostic information is provided, allowing for an adequate, timely treatment of a heart failure condition of the patient.

In particular, as the sensor apparatus is configured to be placed on skin tissue on a patient's extremity, the sensor apparatus may be operative to obtain measurement data relating to peripheral edema of the patient, wherein remote diagnostic signal data as obtained by the sensor apparatus may be correlated with cardiac diagnostic signal data as obtained by the implantable medical device.

In one embodiment, the sensor apparatus comprises a first sensor unity to be placed on a first side of the patient's extremity and a second sensor unit to be placed on a second side of the patient's extremity opposite to the first side. The sensor apparatus thus may comprise different sensor units to be placed on different locations on the patient's extremity, for example on an upper, dorsal side of a patient's foot and on a lower side of the patient's foot.

For example, depending on the placement of a particular sensor unit on the patient's extremity, different diagnostic signals may be obtained, relating for example to a fluid balance in the skin tissue, a hydration of the skin tissue, changes in the interstitial fluid within the skin tissue or relating to blood circulation parameters indicative of e.g. blood pressure.

For example, the first sensor unit may be placed at the top of a patient's foot, the first sensor unit hence being oriented towards the dorsal side of the foot/ankle region. The second sensor unit may, in turn, be arranged at the bottom of the patient's foot, the second sensor unit hence being oriented towards the bottom of the foot/heel/toe region.

By means of the first sensor unit, in particular measurement data may be obtained relating to a quantitative description of edema. By means of the second sensor unit, in turn, in particular measurement data may be obtained indicative of a patient's blood circulatory system.

In one embodiment, the sensor apparatus comprises a mechanical impulse sensor for measuring a measuring quantity related to a response of the skin tissue to a mechanical impulse. The mechanical impulse sensor may for example comprise a mechanical probe and an actuator device for moving the mechanical probe to act onto the skin tissue. The mechanical impulse sensor, using the mechanical probe, may be used to emulate a pitting test as it is conventionally performed by a physician in a clinical test to manually assess an edema. In particular, using the mechanical probe it may be mechanically acted onto the skin tissue in order to deform the skin tissue, wherein deformation characteristics may be assessed in order to quantify edema in the skin tissue.

For example, in one embodiment, the mechanical impulse sensor may be configured to measure at least one of a depression depth of the mechanical probe when acting on the skin tissue, a duration to reach a predefined depression depth of the mechanical probe when acting on the skin tissue, a force applied to the mechanical probe when acting on the skin tissue and/or a resetting duration of the skin tissue to reset after a mechanical impulse of the mechanical probe. By acting on the skin tissue by moving the mechanical probe, hence, a depression is formed on the skin tissue. Based on a displacement of the mechanical probe a depression depth may be determined. Alternatively or in addition, based on monitoring the movement of the mechanical probe a time duration to reach a predefined depression depth may be determined. Alternatively or in addition, a force applied to the mechanical probe may be monitored. Yet alternatively or in addition, after the depression is formed it may be observed, for example based on a movement of the mechanical probe as driven by the resetting movement of the skin tissue, how low it takes the skin tissue to reset to a nondeformed state, wherein the resetting duration may be measured.

By means of the mechanical impulse sensor, a pitting test may be conducted in an automated fashion using the sensor apparatus. This allows for a continuous monitoring of a change in edema condition, wherein data obtained from the mechanical impulse sensor indicative of an edema state may be processed in correlation with cardiac diagnostic signal data obtained by the implantable medical device in order to allow for a diagnosing of a cardiac condition, in particular a heart failure condition.

The actuator device may for example comprise a solenoid for moving the mechanical probe. In another embodiment, the actuator device may comprise an electric motor.

The mechanical probe may have the shape of a piston which is movable along a movement direction with respect to a casing of the mechanical impulse sensor for acting onto skin tissue of the patient's extremity.

The mechanical impulse sensor in particular may be part of the first sensor unit to be placed on the first side of the patient's extremity.

In one embodiment, the sensor apparatus comprises a haptic a sensor comprising an excitation device to output an excitation signal to the skin tissue and a response sensor to sense a physical response signal in response to the excitation signal. The excitation device in particular is configured to cause a sensational signal on the skin tissue, in particular a pain signal for example by a needle or by heat. The response sensor may in particular be a motion sensor in order to sense a physical motion signal in response to the excitation signal.

The haptic sensor in particular may be used to determine a level of neuropathy associated with the patient. It has been found that a heart failure condition may be associated with a neurohormonal overdrive, resulting in exhaustion of endogenous mechanisms associated with peripheral responses. If, by the haptic sensor, it is found that the peripheral nervous system is affected, which may be quantified based on the measurement of a physical response signal in response to an excitation signal output to the skin tissue by the excitation device, this may by indicative of a heart failure condition.

The haptic sensor in particular may be part of the first sensor unit to be placed on the first side of the patient's extremity.

In one embodiment, the sensor apparatus comprises a thermocoupling sensor comprising a heat source to output a heat signal to the skin tissue and a temperature sensor to measure a temperature response to the heat signal. Depending on the water content in the epidermis and dermis, the equivalent thermal conductivity and thermal diffusivity may change. Based on a temperature response as measured by the temperature sensor in response to a heat impulse, hence, the volumetric epidermal water content may be estimated. By means of the heat source, for example a 1° C. change may be induced, and by means of the temperature sensor at some distance to the heat source a corresponding temperature change may be observed indicative of thermal conduction properties of the skin tissue, from which an epidermal water content may be estimated.

The thermocoupling sensor in particular may be part of the first sensor unit to be placed on the first side of the patient's extremity, or may be part of the second sensor unit to be placed on the second side of the patient's extremity.

In one embodiment, the sensor apparatus comprises a bio-impedance sensor to measure a bio-impedance signal on the skin tissue. The bio-impedance sensor may for example be configured to conduct a bio-impedance spectroscopy measurement by conducting impedance measurements by injecting currents into the skin tissue at a multiplicity of different frequencies.

The bio-impedance sensor in particular may be part of the first sensor unit to be placed on the first side of the patient's extremity.

One or multiple different sensors may be combined in the first sensor unit, for example all or some of the above noted sensor devices, for example a mechanical impulse sensor, a haptic sensor, a thermocoupling sensor and/or a bio-impedance sensor.

In one embodiment, the sensor apparatus comprises at least one sensing device to measure a blood circulation parameter on the patient's extremity.

One sensing device may be an optical sensing device configured to measure optical signals in order to derive a blood circulation parameter, relating for example to a blood pressure. The optical sensing device in particular may be a near-infrared optical sensor or a photoplethysmography sensor. The optical sensing device in particular may measure a time delay between an R wave peak and a distal arterial waveform. In particular, the sensing device may measure a pulse arrival time (PAT), which can be assumed to be equal to the sum of a pulse transit time (PTT) and the so-called pre-ejection period (PEP). The pulse transit time is generally inversely related to blood pressure and can be estimated from the relative timing between the proximal and distal wave forms indicative of the arterial pulse. Based on a pulse transit time estimate, blood pressure may be monitored using the sensing device in an automated fashion.

Electrocardiogram signal data as obtained by the implantable medical device herein may provide for reference data for measurement data obtained by the optical sensing device, for example for estimating a pulse arrival time (PAT) and/or pulse transit time (PTT).

The optical sensing device in particular may be part of the second sensor unit to be placed on the second side of the patient's extremity.

Another sensing device may be a ballistocardiography sensing accelerometer, which is configured to measure reactionary forces of the body in response to ventricular ejection of blood into the circulatory system.

The ballistocardiography sensing accelerometer in particular may be part of the second sensor unit to be placed on the second side of the patient's extremity.

Based on output data of e.g. the optical sensing device and the ballistocardiography sensing accelerometer for example a cardiac output or stroke volume may be estimated, and cardiovascular system changes may be monitored.

One or multiple different sensors may be combined in the second sensor unit, for example all or some of the above noted sensor devices, for example an optical sensing device, a ballistocardiography sensing accelerometer, and/or a thermocoupling sensor.

In one embodiment, the sensor apparatus comprises a band device configured for circumferential placement around the patient's extremity. The band device may in particular be an elastic band or strap, which may be placed around the patient's extremity, for example the patient's foot. On the band device the first sensor unit and the second sensor unit, each including one or multiple of the different sensor devices as described above, is arranged such that a sensor arrangement is provided which as a whole may be placed on the patient's extremity and for this may be tightly wrapped around the patient's extremity, such as the patient's foot.

In one embodiment, the band device comprises a measuring device for measuring a change in circumferential length of the band device. For example, for conducting measurements using the sensor apparatus, the band device may be placed tightly around the patient's extremity. If, while the patient carries the sensor apparatus, the circumference of the patient's extremity changes, for example due to a change in edema condition, this may be measured according to a change in the circumferential length of the band device. The measuring device may for example be a deformation sensor configured for measuring a change in distance, for example by using optical fibers.

Measurement signals and data obtained based on such measurement signals of one or of all sensor devices as described in the foregoing may be processed, as remote diagnostic data, in correlation to cardiac diagnostic data as obtained by the implantable medical device. Based on the processing of the data in correlation with one another, information with respect to a cardiac condition, in particular a heart failure condition, may be derived and quantified, for example a change in edema in correlation with a corresponding change in cardiac condition as identified based on electrocardiogram signals.

In one embodiment, the implantable medical device comprises a processing circuitry implementing at least a portion of the processing arrangement for processing signals. The processing circuitry of the implantable medical device in particular may be configured to process data relating to remote diagnostic signals obtained from the sensor apparatus.

Some or all of the processing may be carried out by the implantable medical device. If the processing is carried out by the implantable medical device, data relating to remote diagnostic signals are communicated from the sensor apparatus towards the implantable medical device, which processes the data in correlation with cardiac diagnostic data in order to derive information relating to a cardiac condition, in particular a heart failure condition, for example by correlating information derived from electrocardiogram signal data to information relating to edema in the patient's extremities.

If only some of the processing is carried out by the implantable medical device, data relating to remote diagnostic signals may be communicated for example from the sensor apparatus towards the implantable medical device, which communicates the remote diagnostic signal data and the cardiac diagnostic signal data or information derived therefrom to an external device for further processing, for example within the context of a home monitoring system.

In one embodiment, the implantable medical device comprises a first communication circuitry and the sensor apparatus comprises a second communication circuitry, the first communication circuitry and the second communication circuitry being configured to establish a wireless data communication connection between the implantable medical device and the sensor apparatus. The implantable medical device and sensor apparatus hence may establish a communication connection, such that data may be communicated from the sensor apparatus towards the implantable medical device and/or vice versa.

In another aspect, a sensor apparatus configured to be placed on skin tissue on a patient's extremity comprises a mechanical impulse sensor for measuring a measuring quantity related to a response of the skin tissue to a mechanical impulse, wherein the mechanical impulse sensor comprises a mechanical probe and an actuator device for moving the mechanical probe to act onto the skin tissue.

The mechanical impulse sensor, using the mechanical probe, may be used to emulate a pitting test as it is conventionally performed by a physician in a clinical test to manually assess an edema. In particular, using the mechanical probe it may be mechanically acted onto the skin tissue in order to deform the skin tissue, wherein deformation characteristics may be assessed in order to quantify edema in the skin tissue.

For example, in one embodiment, the mechanical impulse sensor may be configured to measure at least one of a depression depth of the mechanical probe when acting on the skin tissue, a duration to reach a predefined depression depth of the mechanical probe when acting on the skin tissue, a force applied to the mechanical probe when acting on the skin tissue and/or a resetting duration of the skin tissue to reset after a mechanical impulse of the mechanical probe. By acting on the skin tissue by moving the mechanical probe, hence, a depression is formed on the skin tissue. Based on a displacement of the mechanical probe a depression depth may be determined. Alternatively or in addition, based on monitoring the movement of the mechanical probe a time duration to reach a predefined depression depth may be determined. Alternatively or in addition, a force applied to the mechanical probe may be monitored. Yet alternatively or in addition, after the depression is formed it may be observed, for example based on a movement of the mechanical probe as driven by the resetting movement of the skin tissue, how low it takes the skin tissue to reset to a nondeformed state, wherein the resetting duration may be measured.

By means of the mechanical impulse sensor, a pitting test may be conducted in an automated fashion using the sensor apparatus. This allows for a continuous monitoring of a change in edema condition, wherein data obtained from the mechanical impulse sensor indicative of an edema state may be processed in correlation with cardiac diagnostic signal data obtained by the implantable medical device in order to allow for a diagnosing of a cardiac condition, in particular a heart failure condition.

The actuator device may for example comprise a solenoid for moving the mechanical probe. In another embodiment, the actuator device may comprise an electric motor.

The mechanical probe may have the shape of a piston which is movable along a movement direction with respect to a casing of the mechanical impulse sensor for acting onto skin tissue of the patient's extremity.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily under-stood with reference to the following detailed description and the embodiments shown in the drawings. Herein,

FIG. 1 shows a schematic drawing of a system including an implantable medical device implanted in a patient and a sensor apparatus placed on an extremity of the patient;

FIG. 2 shows a schematic drawing of the sensor apparatus placed on an extremity, namely (on or close to) a foot, of a patient;

FIG. 3 shows a schematic drawing of an upper, first sensor unit of the sensor apparatus;

FIG. 4 shows a schematic drawing of a mechanical impulse sensor of the sensor apparatus;

FIG. 5 shows a schematic drawing of a haptic sensor of the sensor apparatus;

FIG. 6 shows a schematic drawing of a thermocoupling sensor of the sensor apparatus;

FIG. 7 shows a schematic drawing of a second sensor unit of the sensor apparatus; and

FIG. 8 shows a schematic drawing of an implantable medical device.

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

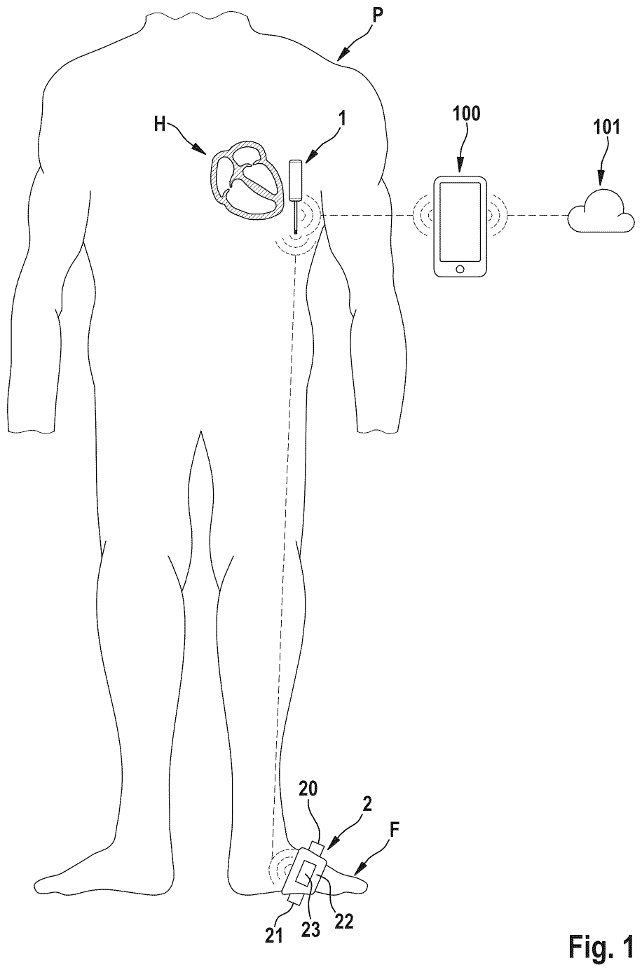

Referring to FIG. 1, in one embodiment a system comprises an implantable medical device 1 implanted (for example subcutaneously) into a patient for serving a therapeutic and/or diagnostic function. The implantable medical device 1 may for example be implanted subcutaneously into a patient P for monitoring cardiac activity of the patient's heart H. The implantable medical device 1, for this, comprises an arrangement of electrode poles which are used to couple to surrounding tissue and to sense electrocardiogram signals originating from the heart H.

The system furthermore comprises an external device 100 external to the patient P and being in communication connection with the implantable medical device 1. The external device 100 in particular may communicate with the implantable medical device 1 in the context of a home monitoring system, the external device 100 being in communication connection with a remote device, such as a remote cloud server device 101 of a home monitoring service center.

In addition, the system comprises a sensor apparatus 2 which is placed on an extremity of the patient P, namely (on or close to) a foot F of the patient P. The sensor apparatus 2 may therefore be located directly on the foot (as shown in FIG. 1), in the ankle area (at or above the heel of the foot) and/or on a lower part of a lower leg (which is connected to the foot). The sensor apparatus 2 is in communication connection with the implantable medical device 1 such that data may be exchanged in between the sensor apparatus 2 and the implantable medical device 1 and may be relayed e.g. via the implantable medical device 1 towards the external device 100 and the remote cloud server device 101.

The implantable medical device 1 may for example be a monitoring device for sensing electrocardiogram signals. In another embodiment, the implantable medical device 1 may be a pacemaker device, a cardioverter defibrillator device, or a sensor device such as a biosensor.

Referring now to FIG. 8, in one embodiment the implantable medical device 1 comprises a housing 10 and an electrode arrangement 11. The electrode arrangement 11 is a sensing arrangement and comprises a multiplicity of electrode poles 110, 111 arranged at different locations on the housing 10 or on one or multiple leads extending from the housing 10 towards a region of interest, such as into the patient's heart. By means of the electrode arrangement 11 the implantable medical device 1 may for example be configured to sense electrical signals and to output electrical stimulation signals, such as pacing signals or the like, for achieving a diagnostic and/or therapeutic function within the patient P.

The implantable medical device 1 comprises a processing circuitry 12 configured for controlling operation of the implantable medical device 1 and for processing signals. In addition, the implantable medical device 1 comprises an energy storage 13, in particular an electrochemical battery. A communication circuitry 14 enables the implantable medical device 1 to establish a communication connection to a communication circuitry 23 of the sensor apparatus 2 and in addition to the external device 100, for example within the context of a home monitoring system.

By means of the system including the implantable medical device 1, in particular a cardiac condition shall be monitored in order to identify a potential heart failure condition.

Clinical presentation of a heart failure condition may generally vary, but is consistently presented with pulmonary congestion, organ perfusion, presence of coronary disease, fluid retention and systemic pressure. In a majority of cases, patients with a heart failure condition present fluid accumulation in the body, in particular in the lower limbs. It has been postulated that fluid retention is responsible for clinical manifestations of heart failure, including fatigue, dyspnea, orthopnea, lower extremity edema, pleural effusions, and ascites.

In a patient with an acute heart failure condition, the presence of a (severe) edema condition may indicate a potential heart failure condition. In addition to edema or fluid overload, measured diagnostics such as nocturnal respiration, nocturnal dyspena and presence of atrial fibrillation are commonly attributed to a heart failure condition. Thus, it is desired in heart failure patients that edema is quantified along with other cardiac parameters, in order to assess a heart failure risk for the patient, and also to enable treatment of the underlying condition with either pharmacologic or electrophysiological procedures in order to minimize the risk and subsequent degradation of cardiac health and function.

Within the system of FIG. 1, the implantable medical device 1 is placed in or in proximity to the patient's heart H, such that cardiac diagnostic signals relating to a cardiac function may be sensed, in particular electrocardiogram signals using an electrode arrangement 11 comprising a multiplicity of electrode poles 110, 111. In addition, remote diagnostic signals are sensed using the sensor apparatus 2 and are communicated to the implantable medical device 1, such that cardiac diagnostic signals as sensed by the implantable medical device 1 and remote diagnostic signals as sensed by the sensor apparatus 2 may be processed in correlation with one another in order to derive information relating to diagnostic data indicative of a potential heart failure condition.

The implantable medical device 1 in particular is configured for sensing cardiac diagnostic measures, such as electrocardiogram signals. In addition, the implantable medical device 1 may comprise a motion sensor 15, as shown in FIG. 8, for example an accelerometer, which may be used to sense motion signals relating to a cardiac motion or to respiratory parameters of the patient. Thus, the motion sensor may also be a respiratory sensor.

In addition, the implantable medical device 1 may comprise e.g. a bio-impedance sensor in order to detect respiratory parameters based on a change of impedance in the thoracic region.

The sensor apparatus 2, in turn, is configured for sensing remote diagnostic signals on an extremity of the patient P, namely (in the shown embodiment) on the foot F of the patient P at a location remote from the patient's heart H.

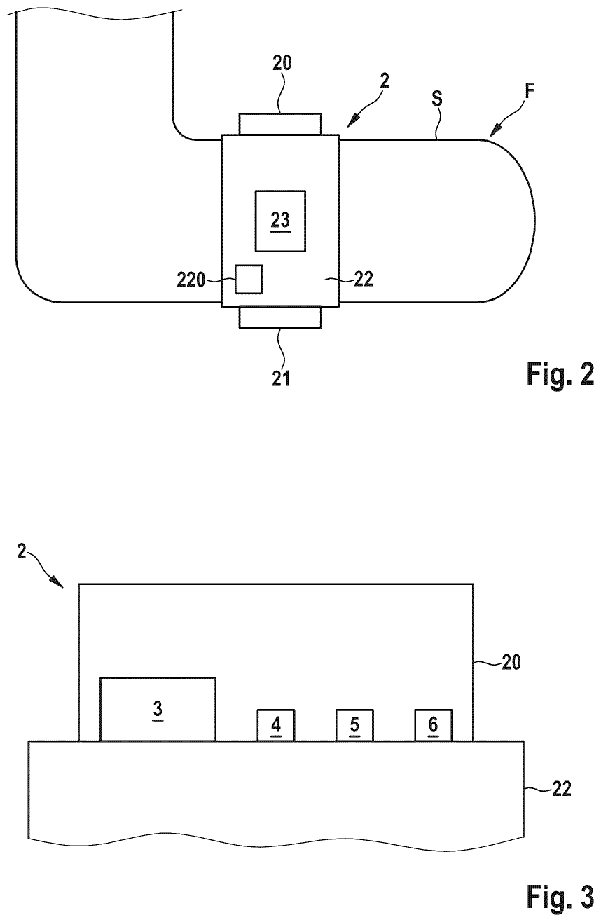

Referring now to FIG. 2, in the shown embodiment the sensor apparatus 2 comprises a band device 22 formed by an elastic band or strap which is placed around the patient's foot F in order to tightly attach the sensor apparatus 2 to the patient's foot F. An upper, first sensor unit 20 is placed on top of the foot F towards the dorsal side. A lower, second sensor unit 21 is placed on a lower side of the foot F opposite to the first sensor unit 20.

The first sensor unit 20 in particular may comprise one or multiple sensor devices to measure signals indicative of edema, in particular indicative of a fluid balance in skin tissue S, hydration of the skin tissue S and changes in the interstitial fluid within the skin tissue S.

The second sensor unit 21 in turn may comprise one or multiple sensors which may be used to measure measurement parameters relating to the blood circulatory system of the patient P, in particular relating to the blood pressure and circulation parameters relating to arterial wall mechanics and wave propagation in the arteries.

A communication circuitry 23 for establishing a data communication with the implantable medical device 1 may be placed within the first sensor unit 20 or within the second sensor unit 21 or outside of either sensor unit 20, 21 on the band device 22.

In addition to sensor devices included in the sensor units 20, 21, a measuring device 220 may be placed on the band device 22 in order to measure a change in circumferential length of the band device 22, for example due to a change in circumference of the patient's foot F.

Referring now to FIG. 3, the first sensor unit 20 may for example comprise a mechanical impulse sensor 3, a haptic sensor 4, a thermocoupling sensor 5 and a bio-impedance sensor 6. The first sensor unit 20 may comprise one, some or all of the sensor devices 3, 4, 5, 6.

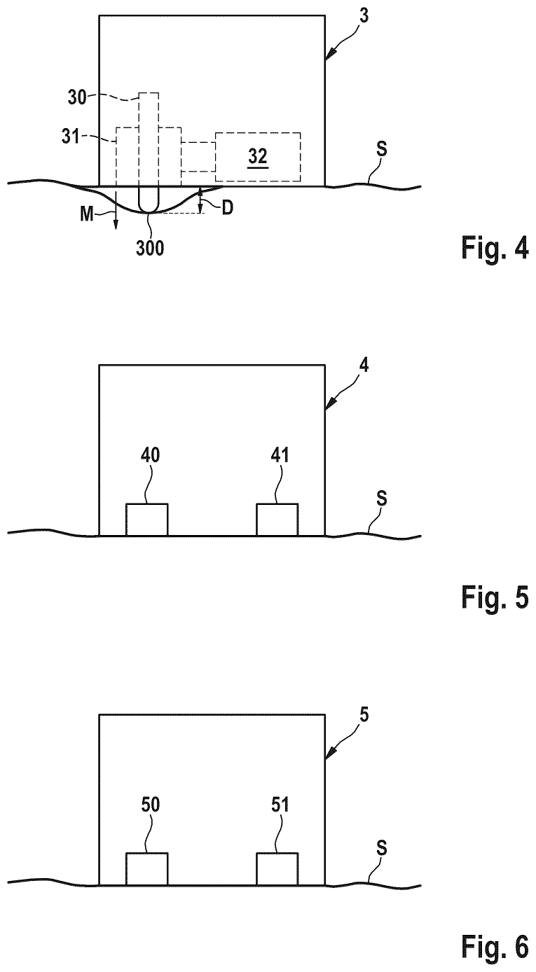

Referring now to FIG. 4, the mechanical impulse sensor 3 may, in one embodiment, comprise a mechanical probe 30 which is movable along a movement direction M by an actuator device 31, for example comprising a solenoid or an electric motor. The mechanical probe 30 is configured to act onto skin tissue S by means of a tip 300, such that by moving the mechanical probe 30 a depression may be formed on the skin tissue S, analogous to a conventional pitting test as it is performed manually by a physician on a patient in a clinical test for edema.

During a test as performed by the mechanical impulse sensor 3, the mechanical probe 30 may be moved in the movement direction M to form a depression on the skin tissue S on which the mechanical impulse sensor 3 is placed. The mechanical probe 30 may for example be moved by the actuator device 31 by applying a defined force. Based on a displacement of the mechanical probe 30 a depression depth D may be measured that is reached after a predefined time duration of moving the mechanical probe 30. Alternatively or in addition, a time duration may be measured that it takes to move the mechanical probe 30 to reach a predefined depth. Yet alternatively or in addition, a force required to reach a predefined depth within a predefined time may be evaluated. Yet alternatively or in addition, for example by assessing a resetting movement of the mechanical probe 30 driven by a resetting of the skin tissue S to a nondeformed state, it may be measured how long it takes the skin tissue S to reset back to its nondeformed state.

Measurement signals may be processed in a processing circuitry 32 to measure a response of skin tissue S to a mechanical deformation in order to derive a quantification score for identifying an edema condition.

Referring now to FIG. 5, a haptic sensor 4 comprises an excitation device 40 and a response sensor 41. The excitation device 40 may be configured to output a sensational signal, such as a signal causing a pain to the patient. The excitation device 40 may for example comprise a needle or a heat source to cause pain on the skin tissue S of the patient P. By means of the response sensor 41 a physical response, in particular a motion response may be sensed. Based on a level of response (none, weak, strong) a level of neuropathy may be assessed and quantified.

Referring now to FIG. 6, a thermocoupling sensor 5 may comprise a heat source 50 and a temperature sensor 51 arranged at some distance to the heat source 50. By means of the heat source 50 a heat signal may be injected into the skin tissue S to cause a local warming of the skin tissue S, for example by 1° C. By means of the temperature sensor 51 a temperature response may be sensed, in order to derive information relating to the water content in the epidermis and dermis, which generally has an influence on the equivalent thermal conductivity and the thermal diffusivity and hence may be assessed based on a measurement of thermal conductivity and diffusivity.

In addition, a bio-impedance sensor 6 of the first sensor unit 20 may be configured for injecting a current signal into the skin tissue S and to measure a voltage signal in response in order to derive impedance information on the skin tissue S. The bio-impedance sensor 6 in particular may be configured for conducting bio-impedance spectroscopy measurements at a multiplicity of frequencies, for example at 100 to 1000, for example 256 different frequencies.

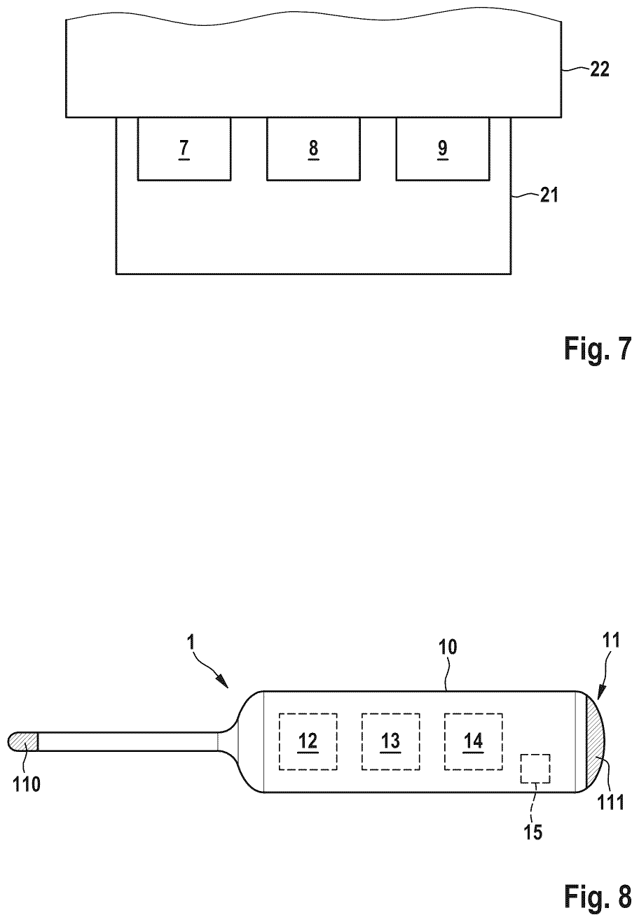

Referring now to FIG. 7, the lower, second sensor unit 21 may comprise an optical sensor 7, for example a near-infrared or photoplethysmogram (PPG) sensor, a ballistocardiography (BCG) sensor 8 and a thermocoupling sensor 9. The second sensor unit 21 may comprise one, some or all of the sensor devices 7, 8, 9.

An optical sensor 7, in particular a near-infrared sensor or a photoplethysmogram (PPG) sensor, may be used to operatively measure a time delay between the R wave peak and a distal arterial waveform, called pulse arrival time (PAT). Based on the pulse arrival time, a pulse transit time (PTT) may be estimated, which inversely is related to blood pressure. Based on a measurement of the optical sensor 7, hence, blood pressure and other parameters relating to cardiac function and cardiac output may be monitored.

As a reference for the optical sensor 7, electrocardiogram signals as sensed by the implantable medical device 1 may be used, such that data obtained by means of the optical sensor 7 may be processed in correlation with data obtained by a sensing arrangement of the implantable medical device 1 to derive information relating to a cardiac output, a stroke volume and cardiovascular system changes.

A ballistocardiography (BCG) sensor 8 comprise an accelerometer to measure a reactionary movement and forces of the patient's body in response to ventricular ejection of blood into the circulatory system.

A thermocoupling sensor 9 measures a thermocoupling to estimate a volumetric epidermal water content, similar to the thermocoupling sensor 5 of the first sensor unit 20.

By operating the sensor apparatus 2 in combination with the implantable medical device 1 and by correlating data obtained from the sensor apparatus 2 with data obtained from the implantable medical device 1, diagnostic information may be derived which may facilitate the monitoring of a heart failure condition and the identification of an acute worsening of a heart failure condition.

Processing of the data may be carried out by the implantable medical device 1, such that measurement data as obtained by the sensor apparatus 2 may be communicated to the implantable medical device 1 for processing within the processing circuitry 12 of the implantable medical device 1.

Some or substantially all of the processing for identifying information relating to a heart failure condition may be carried out by the implantable medical device 1. Processing results as obtained by the implantable medical device 1 may be communicated to the external device 100 within a home monitoring system for further processing of the data in order to allow for a monitoring of a cardiac condition and to facilitate diagnosis for a physician.

A monitoring by the system including the implantable medical device 1 and the sensor apparatus 2 may be carried out in a periodic fashion. For example, measurements by the sensor apparatus 2 may be carried out periodically, for example once or multiple times per day. For example, an edema test using the mechanical impulse sensor 3 may be carried out once each day for a prolonged period of time in order to monitor changes in a cardiac heart failure condition.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device
100 External device
101 Cloud server device
11 Sensing arrangement (electrode arrangement)
110,111 Electrode poles
12 Processing circuitry
13 Energy storage (battery)
14 Communication circuitry
15 Motion sensor
2 Sensor apparatus
20 Upper sensor unit
21 Lower sensor unit
22 Band device
220 Measuring device
23 Communication circuitry
3 Mechanical impulse sensor
30 Mechanical probe
300 Tip
31 Actuator device
32 Processing circuitry
4 Haptic sensor
40 Excitation device
41 Response sensor
5 Thermocoupling sensor
50 Heat source
51 Temperature sensor
6 Bio-impedance sensor
7 Optical sensor (near infrared or photoplethysmogram (PPG) sensor)
8 Ballistocardiography (BCG) sensor
9 Thermocoupling sensor
D Depression depth
F Patient's extremity (foot, ankle, lower leg)
H Heart
M Movement direction
P Patient
S Skin tissue

1. A system for obtaining diagnostic data indicative of a cardiac condition of a patient, comprising:
an implantable medical device comprising a sensing arrangement for sensing a cardiac diagnostic signal;
a sensor apparatus configured to be placed on skin tissue(S) on a patient's extremity and comprising at least one sensor device for sensing a remote diagnostic signal on the skin tissue; and
a processing arrangement for processing data relating to said cardiac diagnostic signal and said remote diagnostic signal to obtain diagnostic data indicative of a cardiac condition.

2. The system according to claim 1, wherein the sensor apparatus comprises a first sensor unit to be placed on a first side of the patient's extremity and a second sensor unit to be placed on a second side of the patient's extremity opposite to the first side.

3. The system according to claim 1, wherein the sensor apparatus comprises a mechanical impulse sensor for measuring a measuring quantity related to a response of the skin tissue to a mechanical impulse.

4. The system according to claim 3, wherein the mechanical impulse sensor comprises a mechanical probe and an actuator device for moving the mechanical probe to act onto the skin tissue.

5. The system according to claim 4, wherein the mechanical impulse sensor is configured to measure at least one of a depression depth of said mechanical probe when acting on the skin tissue, a duration to reach a pre-defined depression depth of said mechanical probe when acting on the skin tissue, a force on said mechanical probe when acting on the skin tissue and/or a resetting duration of the skin tissue to reset after a mechanical impulse of the mechanical probe

6. The system according to one of the laims claim 1, wherein the sensor apparatus comprises a haptic sensor comprising an excitation device to output an excitation signal to the skin tissue and a response sensor to sense a physical response signal in response to said excitation signal.

7. The system according to claim 1, wherein the sensor apparatus comprises a thermocoupling sensor comprising a heat source to output a heat signal to the skin tissue and a temperature sensor to measure a temperature response to said heat signal.

8. The system according to claim 1, wherein the sensor apparatus comprises a bio-impedance sensor to measure a bio-impedance signal on the skin tissue.

9. The system according to one-of-the-p aims claim 1, wherein the sensor apparatus comprises a sensing device to measure a blood circulation parameter on the patient's extremity.

10. The system according to-one of the pre-claims claim 1, wherein the sensor apparatus comprises a band device configured for circumferential placement around the patient's extremity.

11. The system according to claim 10, wherein the band device comprises a measuring device (for measuring a change in circumferential length of the band device.

12. The system according to claim 1, wherein the implantable medical device comprises a processing circuitry implementing at least a portion of said processing arrangement, the processing circuitry being configured to process data relating to said remote diagnostic signal obtained from the sensor apparatus.

13. The system according to claim 1, wherein the implantable medical device comprises a first communication circuitry and the sensor apparatus comprises a second communication circuitry, the first communication circuitry and the second communication circuitry being configured to establish a wireless data communication connection between the implantable medical device and the sensor apparatus

14. A sensor apparatus configured to be placed on skin tissue Son a patient's extremity, the sensor apparatus comprising:
a mechanical impulse sensor for measuring a measuring quantity related to a response of the skin tissue to a mechanical impulse;
wherein the mechanical impulse sensor comprises a mechanical probe and an actuator device for moving the mechanical probe to act onto the skin tissue.

15. The sensor apparatus according to claim 14, wherein the mechanical impulse sensor is configured to measure at least one of a depression depth of said mechanical probe when acting on the skin tissue, a duration to reach a pre-defined depression depth by said mechanical probe when acting on the skin tissue, a force on said mechanical probe when acting on the skin tissue and/or a resetting duration of the skin tissue to reset after a mechanical impulse of the mechanical prober.
